(19) United States
(12) Patent Application Publication
(10) Pub. No.: US 2024/0228940 A1
(43) Pub. Date: Jul. 11, 2024

(54) SYSTEMS AND METHODS FOR MONITORING PACKED-BED CELL CULTURE

(71) Applicant: CORNING INCORPORATED, CORNING, NY (US)

(72) Inventors: Vasiliy Nikolaevich Goral, Painted Post, NY (US); Lori Eileen Romeo, Elmira Heights, NY (US)

(21) Appl. No.: 18/573,720

(22) PCT Filed: Jun. 9, 2022
(86) PCT No.: PCT/US2022/032817

§ 371 Date: Dec. 22, 2023

Related U.S. Application Data
(60) Provisional application No. 63/216,844, filed on Jun. 30, 2021.

(51) Int. Cl. C12M 1/36; C12M 1/00; C12M 1/12

(52) U.S. Cl. CPC C12M 41/48; C12M 25/18; C12M 29/26

(57) ABSTRACT

A bioreactor system for culturing cells is provided. The system includes a cell culture vessel with an interior reservoir. an inlet and an outlet fluidly connected to the reservoir. A fluid flow path supplies fluid to the inlet and receives fluid from the outlet. and a media conditioning vessel is fluidly connected to the cell culture vessel. The system also includes an outlet sensor arranged at the outlet of the cell culture vessel. The outlet sensor can detect a property of cell culture media exiting the cell culture vessel via the outlet, and the bioreactor system can adjust a property of the cell culture media based on the property detected by the outlet sensor.

### CROSS-REFERENCE TO RELATED APPLICATIONS

This application claims the benefit of priority under 35 U.S.C. § 119 of U.S. Provisional Application Ser. No. 63/216,844 filed on Jun. 30, 2021, the content of which is relied upon and incorporated herein by reference in its entirety.

### FIELD OF THE DISCLOSURE

This disclosure general relates to bioreactor systems and related methods for culturing cells. In particular, the present disclosure relates to systems and methods for monitoring and controlling a cell culture within a bioreactor.

### BACKGROUND

In the bioprocessing industry, large-scale cultivation of cells is performed for purposes of the production of hormones, enzymes, antibodies, vaccines, therapeutic proteins, and cell therapies. Cell and gene therapy markets are growing rapidly, with promising treatments moving into clinical trials and quickly toward commercialization. However, one cell therapy dose can require billions of cells or trillions of viruses. As such, being able to provide a large quantity of cell products in a short amount of time is critical for clinical success.

A significant portion of the cells used in bioprocessing are anchorage dependent, meaning the cells need a surface to adhere to for growth and functioning. Traditionally, the culturing of adherent cells is performed on two-dimensional (2D) cell-adherent surfaces incorporated in one of a number of vessel formats, such as T-flasks, petri dishes, cell factories, cell stack vessels, roller bottles, and HYPERStack® vessels. These approaches can have significant drawbacks, including the difficulty in achieving cellular density high enough to make it feasible for large scale production of therapies or cells.

Alternative methods have been suggested to increase volumetric density of cultured cells. These include microcarrier cultures performed in stir tanks. In this approach, cells that are attached to the surface of microcarriers are subject to constant shear stress, resulting in a significant impact on proliferation and culture performance. Another example of a high-density cell culture system is a hollow fiber bioreactor, in which cells may form large three-dimensional aggregates as they proliferate in the interspatial fiber space. However, the cells growth and performance are significantly inhibited by the lack nutrients. To mitigate this problem, these bioreactors are made small and are not suitable for large scale manufacturing

Another example of a high-density culture system for anchorage dependent cells is a packed-bed bioreactor system. In this this type of bioreactor, a cell substrate is used to provide a surface for the attachment of adherent cells. Medium is perfused along the surface or through the semi-porous substrate to provide nutrients and oxygen needed for the cell growth. For example, packed bed bioreactor systems that contain a packed bed of support or matrix systems to entrap the cells have been previously disclosed U.S. Pat. Nos. 4,833,083; 5,501,971; and 5,510,262. Packed bed matrices usually are made of porous particles as substrates or non-woven microfibers of polymer. Such bioreactors function as recirculation flow-through bioreactors. One of the significant issues with such bioreactors is the non-uniformity of cell distribution inside the packed bed. For example, the packed bed functions as depth filter with cells predominantly trapped at the inlet regions, resulting in a gradient of cell distribution during the inoculation step. In addition, due to random fiber packaging, flow resistance and cell trapping efficiency of cross sections of the packed bed are not uniform. For example, medium flows fast though the regions with low cell packing density and flows slowly through the regions where resistance is higher due to higher number of entrapped cells. This creates a channeling effect where nutrients and oxygen are delivered more efficiently to regions with lower volumetric cells densities and regions with higher cell densities are being maintained in suboptimal culture conditions.

Another significant drawback of packed bed systems disclosed in a prior art is the inability to efficiently harvest intact viable cells at the end of culture process. Harvesting of cells is important if the end product is cells, or if the bioreactor is being used as part of a “seed train,” where a cell population is grown in one vessel and then transferred to another vessel for further population growth. U.S. Pat. No. 9,273,278 discloses a bioreactor design to improve the efficiency of cell recovery from the packed bed during cells harvesting step. It is based on loosening the packed bed matrix and agitation or stirring of packed bed particles to allow porous matrices to collide and thus detach the cells. However, this approach is laborious and may cause significant cells damage, thus reducing overall cell viability.

An example of a packed-bed bioreactor currently on the market is the iCellis® by produced by Pall Corporation. The iCellis uses small strips of cell substrate material consisting of randomly oriented fibers in a non-woven arrangement. These strips are packed into a vessel to create a packed bed. However, as with similar solutions on the market, there are drawbacks to this type of packed-bed substrate. Specifically, non-uniform packing of the substrate strips creates visible channels within the packed bed, leading to preferential and non-uniform media flow and nutrient distribution through the packed bed. Studies of the iCellis® have noted a “systemic inhomogeneous distribution of cells, with their number increasing from top to bottom of fixed bed,” as well as a “nutrient gradient . . . leading to restricted cell growth and production,” all of which lead to the “unequal distribution of cells [that] may impair transfection efficiency.” (Rational plasmid design and bioprocess optimization to enhance recombinant adeno-associated virus (AAV) productivity in mammalian cells. Biotechnol. J. 2016, 11, 290-297). Studies have noted that agitation of the packed bed may improve dispersion, but would have other drawbacks (i.e., “necessary agitation for better dispersion during inoculation and transfection would induce increased shear stress, in turn leading to reduced cell viability ” Id.). Another study noted of the iCellis® that the uneven distribution of cells makes monitoring of the cell population using biomass sensors difficult (“. . . if the cells are unevenly distributed, the biomass signal from the cells on the top carriers may not show the general view of the entire bioreactor.” Process Development of Adenoviral Vector Production in Fixed Bed Bioreactor: From Bench to Commercial Scale. Human Gene Therapy, Vol. 26, No. 8, 2015).

In addition, because of the random arrangement of fibers in the substrate strips and the variation in packing of strips between onepacked bed and another of the iCellis®, it can be difficult for customers to predict cell culture performance, since the substrate varies between cultures. Furthermore, the packed substrate of the iCellis® makes efficiently harvesting cells very difficult or impossible, as it is believed that cells are entrapped by the packed bed.

Regardless of the platform used, the earlier stages of process development require users to have information for better understanding of cell behavior, virus production, and culture progress. The upstream bioreactor process development requires the identification of critical parameters and quality features as well as the parameter definition and their connection to the final product. Understanding what these parameters are and how they might scale with higher-density or larger systems is important for process development and efficiency.

Upstream bioprocess production also goes through good manufacturing process (GMP) regulations as well as requirements referred to process analytical technology (PAT). PAT is regarded as a tool for the design, analyses and control of production processes. The final product quality can be ensured through the measurement of process parameters and product characteristics. This can include extensive online culture process monitoring, which provides a useful tool for process characterization and the detection of process changes. Relevant parameters for packed bed bioreactor process characterization and control are pH, temperature, dissolved oxygen or oxygen delivery (DO2), and carbon dioxide (CO₂). However, one of the identified drawbacks of packed bed bioreactors is the difficulty in taking substrate samples to directly assess the state of the cells and the overall cell culture progress. Taking substrate samples risk contaminating the entire culture or, in the case of non-uniform platforms, providing misleading or inaccurate data.

While manufacturing of viral vectors for early-phase clinical trials is possible with existing platforms, there is a need for a platform that can produce high-quality product in greater numbers in order to reach late-stage commercial manufacturing scale. In addition, there is a need for systems and methods that enable collection of specific measurable parameters from the cell culture during a bioreactor run to have better real time control of aspects of the culture process, and to detect and diagnose abnormal culture conditions.

### SUMMARY

According to an embodiment of this disclosure, a packed-bed bioreactor system for culturing cells is provided. The system includes: a cell culture vessel including at least one interior reservoir, an inlet fluidly connected to the reservoir, and an outlet fluidly connected to the reservoir; a fluid flow path to supply fluid to the inlet and receive fluid from the outlet; a media conditioning vessel fluidly connected to the cell culture vessel; an outlet sensor arranged at the outlet of the cell culture vessel. The outlet sensor can detect a property of cell culture media exiting the cell culture vessel via the outlet, and the bioreactor system can adjust a property of the cell culture media based on the property detected by the outlet sensor.

According to an aspect of some embodiments, the property detected by the outlet sensor can be at least one of pH, temperature, dissolved gas level, and nutrient level of the cell culture media. The dissolved gas can be at least one of air, dissolved oxygen, or carbon dioxide. The property of the cell culture media that is adjusted can be at least one of pH, temperature, dissolved gas level, nutrient level, and flow rate of the cell culture media. The system can further include a pump to control a flow rate in the fluid flow path. The bioreactor system controls the pump to adjust the flow rate based on the property detected by the outlet sensor. As an aspect of some embodiments, te bioreactor system is configured to increase the flow rate when the amount of the dissolved gas decreases below a predetermined level.

According to one or more embodiments, a bioreactor system for culturing cells is provide that includes a cell culture vessel having at least one interior reservoir, an inlet fluidly connected to the reservoir, and an outlet fluidly connected to the reservoir; an outlet sensor arranged at or near the outlet of the cell culture vessel; and a pump to supply fluid to the inlet. The pump can adjust a perfusion rate of fluid in the bioreactor system based on a signal from the outlet sensor. In an aspect of some embodiments, the outlet sensor can measure at least one of pH, temperature, dissolved gas level, and nutrient level of the fluid exiting the cell culture vessel.

According to one or more embodiments, a method of controlling a bioreactor system for culturing cells is provided. The method includes perfusing cell culture media through a cell culture vessel; measuring an outlet property of the cell culture media using an outlet sensor at an outlet of the cell culture vessel, and adjusting an inlet property of the cell culture media based on the property measured. The outlet property measured is at least one of pH, temperature, dissolved gas level, and nutrient level of the fluid. The inlet property is at least one of pH, temperature, dissolved gas level, nutrient level, and flow rate of the cell culture media. The method can further include conditioning the cell culture media in a media conditioning vessel to adjust at least one of pH, temperature, dissolved gas level, and nutrient level of the cell culture media before it enters the cell culture vessel. In an aspect of some embodiments, the method further includes controlling a pump to adjusting the flow rate of cell culture media through the cell culture vessel. The method can further include measuring over time a level of dissolved oxygen in the cell culture media using the outlet sensor and calculating a rate of change of oxygen consumption by the cell culture over time; and using the rate of change of oxygen consumption to determine a quality of health of the cell culture.

### BRIEF DESCRIPTION OF THE DRAWINGS

FIG. 1 is a schematic representation of a cell culture system, according to one or more embodiments.

FIG. 2 shows an operation for controlling a perfusion flow rate of a cell culture system, according to one or more embodiments.

FIG. 3 is a graph of bioreactor perfusion flow rate and oxygen concentration over time during an example bioreactor run using a bioreactor system according to FIG. 1, according to one or more embodiments.

FIG. 4A is a graph of the dissolved oxygen concentration over time during the bioreactor run of FIG. 3.

FIG. 4B is a graph of the pH over time during the bioreactor run of FIG. 3.

FIG. 4C is a graph of the media conditioning temperature over time during the bioreactor run of FIG. 3.

FIG. 5 is a graph of the oxygen consumption of the packed bed cell culture over time during the bioreactor run of FIG. 3, including the slope a of the curve.

FIG. 6 is a graph of the slope a versus cell seeding density of the bioreactor, according to one or more embodiments.

FIG. 7 is a graph of the slope a versus cell culture substrate surface area, according to one or more embodiments.

FIG. 8 is a graph of the cell harvest yield versus the slope a, according to one or more embodiments.

### DETAILED DESCRIPTION

Various embodiments of the disclosure will be described in detail with reference to drawings, if any. Reference to various embodiments does not limit the scope of the invention, which is limited only by the scope of the claims attached hereto. Additionally, any examples set forth in this specification are not limiting and merely set forth some of the many possible embodiments of the claimed invention.

Embodiments of this disclosure include systems and methods for monitoring and controlling the cell culture. This disclosure describes systems and methods to collect specific signal signatures during bioreactor run to have better real time control of critical aspects and to detect and diagnose abnormal culture conditions. Identified signature parameters of the cell culture described in this disclosure can be used as a tool for process analytical technology implementation and for online monitoring of upstream process. As a result, the optimized cell culture production processes can be established by development of routine and reproducibility of the signature operating parameters.

According to embodiments of this disclosure, bioreactor systems and methods are provided for monitoring the state of a cell culture in the bioreactor system during a cell culture run. In particular, embodiments describe a bioreactor system having an outlet sensor at the outlet of a cell culture bioreactor or vessel, as well as systems capable of real-time signal collection and real-time process of signals from this and/or other sensors, and methods of cell culture using such systems. For example, methods include using such sensor signals as trigger points for important cell culture process steps, or for predicting the expected or assessing the current health of a cell culture for a certain bioreactor size or seeding density over time. The advantages of these systems and methods include the ability to actively monitor the bioreactor state in real time without the need to perform physical sampling of the packed bed substrate for off-line analysis. Continuous monitoring of bioreactor state will also allow end users to actively adjust the bioprocess steps that are dependent on the progression of culture processes inside the packed bed bioreactor. The ability to characterize and log the progression of bioprocess run will further allow end users to monitor and record batch to batch consistency of the process. This type of tracking of progression and consistency among cell culture runs can be incredibly advantageous.

In conventional large-scale cell culture bioreactors, different types of packed bed bioreactors have been used. Usually these packed beds contain porous matrices to retain adherent or suspension cells, and to support growth and proliferation. Packed-bed matrices provide high surface area to volume ratios, so cell density can be higher than in the other systems. However, the packed bed often functions as a depth filter, where cells are physically trapped or entangled in fibers of the matrix. Thus, because of linear flow of the cell inoculum through the packed bed, cells are subject to heterogeneous distribution inside the packed-bed, leading to variations in cell density through the depth or width of the packed bed. For example, cell density may be higher at the inlet region of a bioreactor and significantly lower nearer to the outlet of the bioreactor. In another example, non-uniformities in the packed bed create a channeling effect in which cell culture media preferentially flows in certain areas of the bed while be restricted from reaching other areas of the bed, again leading to non-uniform cell distribution and nonuniform or inconsistent medium or nutrient distribution. This non-uniform distribution of the cells inside of the packed-bed significantly hinders scalability and predictability of such bioreactors in bioprocess manufacturing, and can even lead to reduced efficiency in terms of growth of cells or viral vector production per unit surface area or volume of the packed bed.

Another problem encountered in packed bed bioreactors disclosed in prior art is the channeling effect, described above. Due to random nature of packed nonwoven fibers, the local fiber density at any given cross section of the packed bed is not uniform. Medium flows quickly in the regions with low fiber density (high bed permeability) and much slower in the regions of high fiber density (lower bed permeability). The resulting non-uniform media perfusion across the packed bed creates the channeling effect, which manifests itself as significant nutrient and metabolite gradients that negatively impact overall cell culture and bioreactor performance. Cells located in the regions of low media perfusion will starve and very often die from the lack of nutrients or metabolite poisoning. Cell harvesting is yet another problem encountered when bioreactors packed with non-woven fibrous scaffolds are used. Due to packed-bed functions as depth filter, cells that are released at the end of cell culture process are entrapped inside the packed bed, and cell recovery is very low. This significantly limits utilization of such bioreactors in bioprocesses where live cells are the products. Thus, the non-uniformity leads to areas with different exposure to flow and shear, effectively reducing the usable cell culture area, causing non-uniform culture, and interfering with transfection efficiency and cell release.

To address these and other problems of existing cell culture solutions, embodiments of the present disclosure provide bioreactor systems, cell growth substrates, matrices of such substrates, and methods using such bioreactor systems and substrates that enable efficient and high-yield cell culturing for anchorage-dependent cells and production of cell products (e.g., proteins, antibodies, viral particles). Embodiments include a porous cell-culture matrix made from an ordered and regular array of porous substrate material that enables uniform cell seeding and media/nutrient perfusion, as well as efficient cell harvesting. Embodiments also enable scalable cell-culture solutions with substrates and bioreactors capable of seeding and growing cells and/or harvesting cell products from a process development scale to a full production size scale, without sacrificing the uniform performance of the embodiments. For example, in some embodiments, a bioreactor can be easily scaled from process development scale to product scale with comparable viral genome per unit surface area of substrate (VG/cm²) across the production scale. The harvestability and scalability of the embodiments herein enable their use in efficient seed trains for growing cell populations at multiple scales on the same cell substrate. In addition, the embodiments herein provide a cell culture matrix having a high surface area that, in combination with the other features described, enables a high yield cell culture solution. In some embodiments, for example, the cell culture substrate and/or bioreactors discussed herein can produce 10¹⁶ to 10¹⁸ viral genomes (VG) per batch.

Embodiments of this disclosure can achieve viral vector platforms of a practical size that can produce viral genomes on the scale of greater than about 10¹⁴ viral genomes per batch, greater than about 10¹⁵ viral genomes per batch, greater than about 10¹⁶ viral genomes per batch, greater than about 10¹⁷ viral genomes per batch, or up to or greater than about g 10¹⁶ viral genomes per batch. In some embodiments, productions is about 10¹⁵ to about 10¹⁸ or more viral genomes per batch. For example, in some embodiments, the viral genome yield can be about 10¹⁵ to about 10¹⁶ viral genomes or batch, or about 10¹⁶ to about 10¹⁹ viral genomes per batch, or about 10¹⁶-10¹⁸ viral genomes per batch, or about 10¹⁷ to about 10¹⁹ viral genomes per batch, or about 10¹⁸ to about 10¹⁹ viral genomes per batch, or about 10¹⁸ or more viral genomes per batch.

In addition, the embodiments disclosed herein enable not only cell attachment and growth to a cell culture substrate, but also the viable harvest of cultured cells. The inability to harvest viable cells is a significant drawback in current platforms, and it leads to difficulty in building and sustaining a sufficient number of cells for production capacity. According to an aspect of embodiments of this disclosure, it is possible to harvest viable cells from the cell culture substrate, including between 80% to 100% viable, or about 85% to about 99% viable, or about 90% to about 99% viable For example, of the cells that are harvested, at least 80% are viable, at least 85% are viable, at least 90% are viable, at least 91% are viable, at least 92% are viable, at least 93% are viable, at least 94% are viable, at least 95% are viable, at least 96% are viable, at least 97% are viable, at least 98% are viable, or at least 99% are viable. Cells may be released from the cell culture substrate using, for example, trypsin, TrypLE, or Accutase.

According to one or more embodiments, a cell culture bioreactor can include a cell culture substrate within the bioreactor vessel. The substrate can be deployed in a packed bed bioreactor configuration, or in other configurations within a three-dimensional culture chamber of the bioreactor vessel. Due to contamination concerns, the vessel can be a single-use vessel that can be disposed of after use.

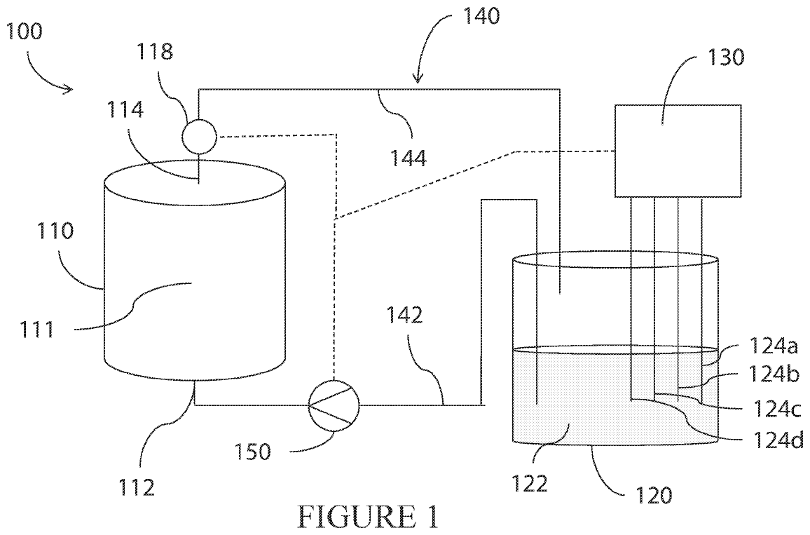

As shown in FIG. 1, embodiments of this disclosure include a bioreactor system 100 for culturing cells in a cell culture vessel 100. The cell culture vessel includes an inlet 112 and an outlet 114 that are fluidly connected to an interior reservoir 111 of the cell culture vessel 110. The interior reservoir 111 contains a space for containing and culturing cells, and may also include a cell culture substrate (not shown) on which adherent-based cells can be cultured. In some embodiments, the inlet 112 is located at one end of the cell culture vessel 110 for the input of media, cells, and/or nutrients into the cell culture vessel 110, and the outlet 114 is located at the opposite end for removing media, cells, or cell products from the cell culture vessel 110. The substrate within the interior reservoir can take many forms, some of which are discussed herein by way of example. Some embodiments may use one or both of the inlet 112 and outlet 114 for flowing media, cells, or other contents both into and out of the cell culture vessel 110. For example, inlet 112 may be used for flowing media or cells into the cell culture vessel 110, during cell seeding, perfusion, and/or culturing phases, but may also be used for removing one or more of media, cells, or cell products through the inlet 112 in a harvesting phase. Thus, the terms “inlet” and “outlet” are not intended to restrict the function of those openings, but should generally be understood to mean the ports used for inletting and outletting, respectively, of fluid during the regular course of cell growth. An outlet sensor 118 is provided at the outlet 114 of the cell culture vessel 110. As used herein, “at the outlet” can mean a sensor providing in-line sensing of a fluid flow path that receives media from the outlet 114 and returns the media to another part of the system (e.g., a media conditioning vessel), or it can mean a sensor provided within the cell culture vessel 110 but preferably after the cell culture substrate, packed bed, or other cell culture zone within the cell culture vessel 110. In this way, the outlet sensor 114 can detect a property of media after it has passed through the packed bed, cell culture substrate, or other cell culture zone.

According to embodiments, the outlet sensor 118 can be an in-line sensor integrated into, coupled to, and/or removably attached to the outlet 114 and/or a flow path connection (e.g., tubing, piping, or conduit) connected to the outlet 114. The outlet sensor 118 can, for example, be a disposable in-line sensor. By providing an in-line senor at the outlet 114 or in tubing connected to the outlet 114, the outlet sensor 118 can sense properties of the media without disturbing the cell culture within the bioreactor and/or without stopping the cell culture (e.g., while perfusing media through the bioreactor).

The system further includes a media conditioning vessel (MCV) 120 that can hold and condition cell culture media 122. A fluid flow path 142, 144 delivers conditioned media 112 from the MCV 120 to the cell culture vessel 110, and returns used media from the cell culture vessel 110 to the MCV 120. The MCV 120 can be coupled with a plurality of sensors and/or conditioning components 124a, 1246, 124c, 124d used to sense properties of the cell culture media and to adjust or condition that media, as needed during the cell culture. These include but are not limited to dissolved gas (e.g., O₂, air, CO₂, N₂) sensors and supplies, pH sensors, oxygenator/gas sparging unit, temperature probes and temperature control devices, and nutrient addition and base addition ports. A gas mixture supplied to sparging unit can be controlled by a gas flow controller for N₂, O₂, and CO₂ gasses. The media conditioning vessel 120 can also contain an impeller for media mixing.

The system can also include a media conditioning control unit 130, operatively connected to the plurality of sensors and/or conditioning components 124a, 124b, 124c, 124d to process signals detected from those sensors and/or to control the conditioning components to condition the media 122 within the MCV 120. The media conditioning control unit 130 can also be operatively connected to a pump 150 to control the pump 150 and thus control the rate of fluid flow through the fluid flow path 142, 144 and the perfusion through the cell culture vessel 110. Alternatively, the pump 150 and outlet sensor 118 can be connected directly or connected via a perfusion control unit separate from the media conditioning control unit 130. In some embodiments herein, a peristaltic pump is used, but other pump types are possible. As shown in FIG. 1, the media conditioning vessel 120 is provided as a vessel that is separate from the bioreactor vessel 110. This can have advantages in terms of being able to condition the media separate from where the cells are cultured, and then supplying the conditioned media to the cell culture space. However, in some embodiments, media conditioning can be performed within the bioreactor vessel 110.

In some embodiments, the media conditioning control unit 130 can be used to maintain a steady or desired level of various parameters of the cell culture media 122 within the MCV 120, thus maintaining the bulk media 122 at specific temperature, oxygen saturation level, pH, and CO₂ concentration. For example, for a given cell line or stage of the cell culture process, it may be desirable for the cell culture media 122 to have a certain temperature, pH, dissolved gas content, or nutrient level for optimal cell health and/or growth. The media 122 from the media conditioning vessel 120 is delivered to the cell culture vessel 110 via the inlet 112, which may also include an injection port for cell inoculum to seed and begin culturing of cells. The cell culture vessel 110 may also include the outlet 114 through which the cell culture media 122 exits the vessel 110. In addition, cells or cell products may be output through the outlet 114. To analyze the contents of the outflow from the cell culture vessel 110, the outlet sensor 118 is provided. As described above, the media conditioning control unit 130 may receive a signal from the outlet sensor 118 (e.g., an O₂ sensor) and, based on the signal, adjust the fluid flow through the cell culture vessel 110 by sending a signal to a pump 150 (e.g., peristaltic pump) upstream of the inlet 112 of the cell culture vessel 110. Thus, based on one or a combination of factors measured by the outlet sensor 118, the pump 150 can control the flow into the cell culture vessel 110 to obtain the desired cell culturing conditions. Because the cell culture media 122 within the MCV 120 can be maintained at a desired state, the changing of the flow rate can effectively address any need of the cells within the cell culture vessel 110. For example, because cell culture media 122 leaving the MCV 120 is conditioning for optimal performance, the media entering via inlet 112 should meet the optimal requirements for the media. If the outlet sensor 118 detects a less than desirable level in the cell culture media existing the cell culture vessel 110 at the outlet 114, that can mean, for example, that the cells in the culture have consumed some amount of dissolved gas (e.g., oxygen) or cell nutrients in the media, and at least some cells (i.e., those near the outlet where the media is most depleted) are not being cultured optimally. Therefore, if, for example, the level of dissolved oxygen in the cell culture media at the outlet sensor 118 is lower than optimal (e g., for a given cell type, stage of culture, etc.), the perfusion flow rate can be increased to supply a higher rate of the conditioned media, which should then result in all cells (even those near the outlet) being cultured under optimal conditions.

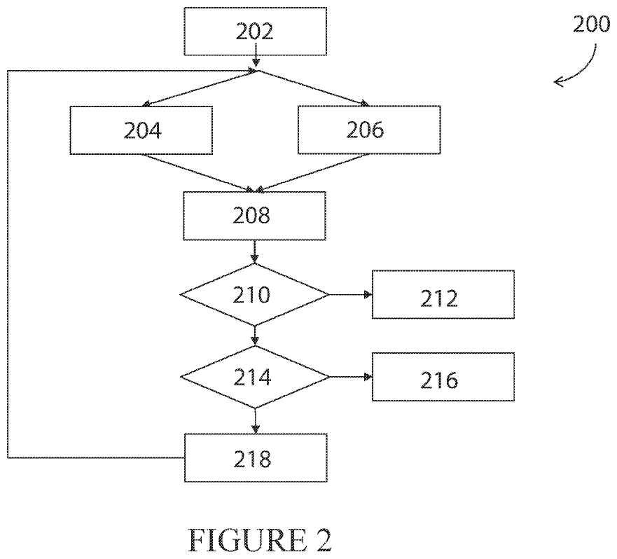

The media perfusion rate is controlled by the media conditioning control unit 130 that collects and compares sensors signals from media conditioning vessel 120 and sensors 124a-124d in the MCV 120, as well as the outlet sensor 118. Because of the pack flow nature of media perfusion through a packed bed substrate in the cell culture vessel 110, nutrients, pH and oxygen gradients are developed along the packed bed. The perfusion flow rate of the bioreactor can be automatically controlled by the media conditioning control unit 130 operably connected to the pump 150. This control scheme is represented in the flow diagram of FIG. 2. In the sensing and control process 200 shown in FIG. 2, at step 202, optimal conditions are predetermined through a round of bioreactor optimization runs. These optimal conditions include minimum pH, minimum oxygen level, and nutrient (e.g., glucose) at the outlet sensor 118, and the pH, oxygen level, and nutrient (e.g., glucose) in the MCV 120. These parameters are provided by way of example, and a person of ordinary skill in the art could understand that other parameters may be relevant for a given application (e.g., temperature). The pH and oxygen levels in the MCV 120 are controlled independently based on inputs from the respective sensors located in the MCV 120. The nutrient (e.g., glucose) level is maintained in the MCV 120 based in part on a signal from the outlet sensor 118, such that the nutrient level in the MCV 120 remains greater than a nutrient level detected by the outlet sensor 118. During the cell culture run, step 204 and 206 are conducted in parallel. In step 204, the outlet sensor 118 is used to measure conditions at the cell culture vessel 110 outlet 114 (e.g., pH, O₂, and glucose). In step 206, sensors 124a-124d are used to measure conditions in the MCV 120 (e.g., pH, O₂, and glucose). In step 208, the perfusion pump 150 is controlled by the control unit based on input from both steps 202 and 204. In step 210, it is determined if the pH at the outlet sensor 118 is greater than the minimum pH determined in step 202: if the oxygen at the outlet sensor 118 is greater than the minimum oxygen level determined in step 202; and if the nutrient level in the MCV 120 is greater than the nutrient level at the outlet sensor 118, and whether the nutrient level at the outlet sensor is greater than the minimum level determined in step 202. If all of these conditions are met, the perfusion by the pump is continued at the present flow rate (step 212). If those conditions are not met, step 214 asks whether the current perfusion rate is less than or equal to the max flow rate. If itis not, then the system reevaluates minimum pH, O₂, and glucose at the outlet 114, or increases the nutrient level in the MCV 120. However, if the current perfusion rate is less than the max flow rate, step 218 dictates that the perfusion flow rate be increased. The sensing and control scheme 200 returns to the top of the chart in FIG. 2 for steps 204 and 206

According to embodiments of this disclosure, it is therefore possible to directly measure nutrient and/or oxygen consumption of cells within the cell culture vessel and to respond in a way that maintains desired conditions for the cells. For example, during the cell culture run, the media conditioning control unit 130 is preprogrammed to maintain a specific level of oxygen saturation in the bulk media volume relative to the atmospheric saturation, where that level in the MCV is measured by the sensors 124a-124d. Placement of second sensor (outlet sensor 118) at the bioreactor outlet 114 measures oxygen saturation level in the media just as it leaves the cell culture vessel. Using these sensors and controls, a constant oxygen depletion level can be maintained within the range of physiological conditions by automatic adjustment of perfusion flow rate.

According to some embodiments of this disclosure, systems and methods are provided for improved process monitoring that can accelerate the process development for cell culture protocols and improve efficiency and reproducibility of the cell culture process. The ability to characterize and log the progression of these bioprocess runs will allow the end user to monitor and record batch to batch consistency of the process. Relevant parameters for the process characterization are cell growth, cell quality, medium conditions (temperature, pH, pO2, and pCO2) as well as metabolite concentrations (glucose, lactate, glutamine and ammonium). Temperature, pH, pO2, and pCO2 of bulk media are routinely controlled online in cell culture, but online monitoring of these and other process parameters in dynamic systems is not done today. Thus, embodiments of this disclosure provide systems and methods to obtain, e.g., the oxygen consumption parameter in a packed bed perfusion bioreactor and demonstrate that such parameter is characteristic for a given bioprocess, and therefore can be used as a signature parameter for a given bioprocess.

As discussed above, FIG. 1 presents a schematic of bioreactor system (e.g., a packed bed perfusion bioreactor). Cell culture media that enters the cell culture vessel 110 through the inlet 112 can have 100% atmospheric oxygen saturation. Alternatively, according to Henry's law, concentration of a gas in liquid phase is equal to Henry's law constant (k) multiplied by the partial pressure of that gas in the gas phase, therefore oxygen saturation can be presented as concentration of oxygen in cell culture media and equal to 204 μM at 100% saturation at normal atmospheric pressure. During media passage through the cell culture vessel, dissolved oxygen is being used by the immobilized cells and its concentration in the cell culture media drops. Different cell types have different oxygen consumption rates. But a bioreactor system with a sensing and control system of this disclosure allows a user to run the process with specified oxygen concentration at the bioreactor outlet, measured by the outlet sensors 118 and the media conditioning and perfusion control system operates according to logic presented in the flow diagram of FIG. 2.

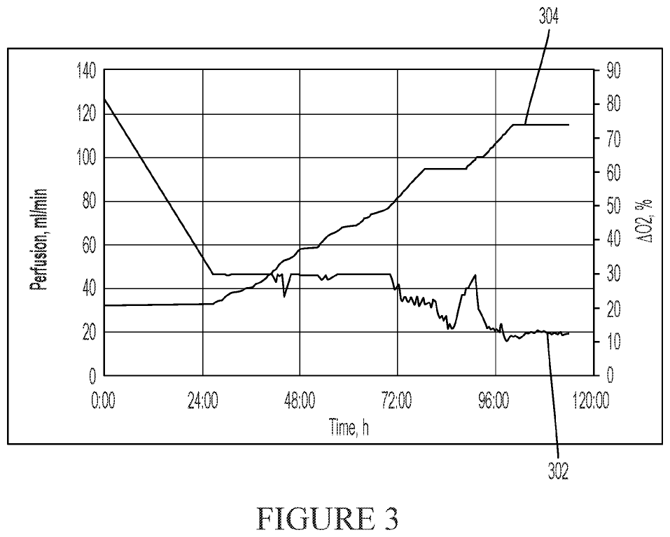

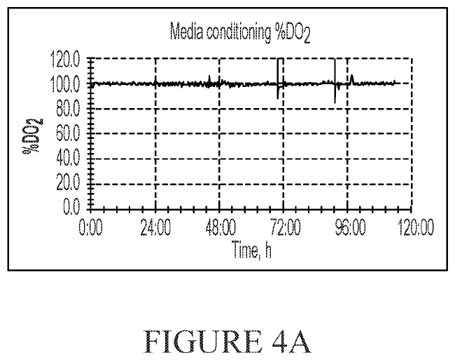

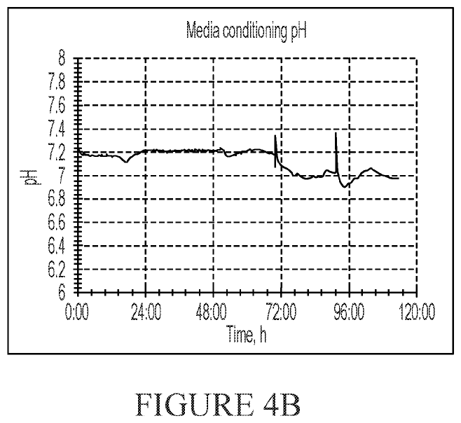

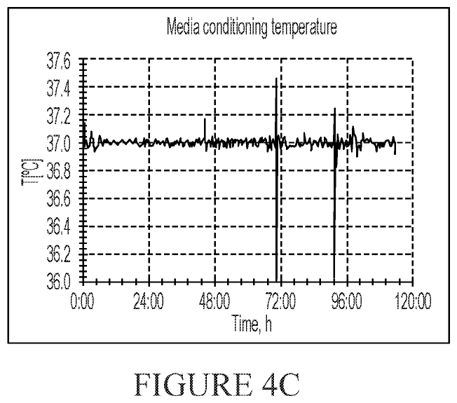

To illustrate this sensing and control of the bioreactor system, some examples will be presented. Specifically, FIG. 3 shows a typical graph of percent dissolved oxygen (302) over time during a bioreactor run as measured by an outlet sensor 118, and the corresponding perfusion rate (304) (ml/min) of the media in the system. Flow rate was controlled automatically by a peristaltic perfusion flow control unit. In this example, the bioreactor is seeded with the cells at time 0:00 hours and the user set a minimal oxygen saturation level of media at the outlet sensor 118 to 30%. The initial media perfusion flow rate was set to 33 ml/min. Inoculation cells are then provided into the bioreactor system and begin to attach to the packed bed substrate and proliferate. Accordingly, oxygen consumption increases and saturation level of the media at outlet decreases to ˜30% a.s. at 26 hours post-seeding. As a result, the control system automatically increases the perfusion flow rate to maintain that minimum 30% oxygen saturation level at the bioreactor outlet 114. At 72 hours post-inoculation, the user decreased the setting of the minimum outlet oxygen saturation level from 30% to 15% and the cell culture proceeded in automatic mode. It should be noted that medium conditions were independently maintained in a media conditioning vessel by the media conditioning control unit. Examples of media conditioning vessel parameters are presented in FIGS. 4A, 4B, and 4C. Specifically, FIG. 4A shows the percentage of oxygen in the media of the MCV over time. FIG. 4B shows the pH of that media over time, and FIG. 4C shows the temperature of that media over time.

As mentioned above, embodiments include the real time processing of signals and control of a bioreactor system, and the development of a characteristic signal signature of a specific bioreactor run, which can be used as an analytical tool and to compare and validate independent bioreactor runs. Thus, characteristic signal signature can be used to evaluate the health of cells cultured inside the cell culture vessel and make decisions regarding the next process steps to occur during the bioreactor run. For example, as discussed above, FIG. 3 shows recorded oxygen saturation concentration over time at the bioreactor outlet 114 during the cell culture process. The oxygen concentration level at the bioreactor outlet dropped from ˜82% at time point 0 hours to about 30% during the first 26 hours of the bioreactor run. While this occurred, the oxygen concentration at the bioreactor inlet 112 was kept constant, as shown by the value detected in the MCV 120 in FIG. 4A. Using the oxygen concentration at inlet and outlet, an oxygen consumption rate for the cells in culture can be determined using Equation 1:

$$Total oxygen consumption in packed bed ( \frac{\% a . s .}{min} ) = Pump flow rate ( \frac{ml}{min} ) * ( 100 - Oxygen saturation level at bioreactor outlet , \% ) . \qquad Equation ( 1 )$$

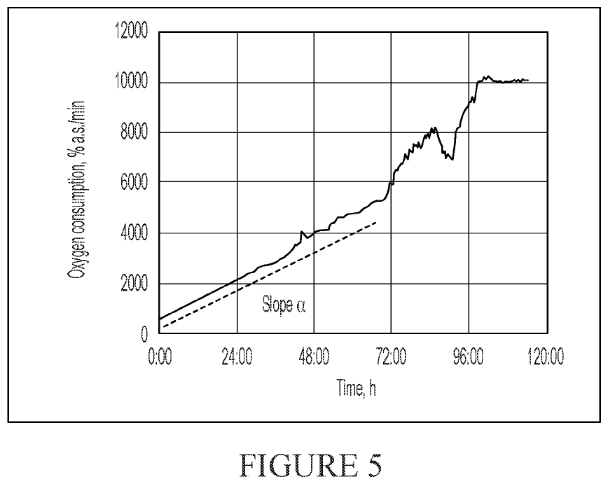

This oxygen consumption rate is shown in FIG. 5 (expressed in % a.s./min) over time (in hours). FIG. 5 also shows a dotted line representing the approximate slope, α, of the line, which can be used as the characteristic signal signature of the bioreactor run. In other words, the value of the data's slope α in FIG. 5 directly reflects the cell culture progression inside the bioreactor system. This value can be used as a process analytical tool to control and describe the upstream bioprocess. The examples below demonstrate that the slope α from graphs similar to FIG. 5 directly relates to the health of the cell culture and from the biomass inside packed bed matrix.

To illustrate the use of the parameter α, multiple cell culture runs were performed using bioreactor systems having differently sized cell culture substrate, bed heights, and cell seeding densities. Table 1 summarizes parameters of the seven cell culture runs used.

TABLE 1
Summary of 7 bioreactor system runs, including height of the
packed bed (cm), total cells seeded (millions of cells), total
packed bed surface area (cm²), seeding density (cells/cm²),
total harvested cells (billions of cells), viability of harvested
cells (%), harvested density (cells/cm²), maximum perfusion flow
rate (ml/min), maximum O₂ consumption (AU), and slope α.

| | | Total | | | | | | | | |
|---|---|---|---|---|---|---|---|---|---|---|
| | Height | cells | Total | | | | Harvested | Max. | | |
| | of the | seeded, | packed bed | Seed | Total | | density, | perfusion | Max. O₂ | |
| | packed | millions | surface | density, | harvested cells, | | thousand | flow, | consumption, | Slope |
| # | bed, cm | cells | area, cm² | cells/cm² | billions cells | Viability | cells/cm² | ml/min | AU | α |
| 1 | 2.7 | 151 | 6780 | 22,222 | 3.12 | 98% | 460K | 91 | 5400 | 42 |
| 2 | 2.7 | 151 | 6780 | 22,222 | 2.47 | 92% | 364K | 100 | 6000 | 34.62 |
| 3 | 2.7 | 151 | 6780 | 22,222 | 1.68 | 96% | 248K | 70 | 4000 | 19 |
| 4 | 5.4 | 302 | 13560 | 22,227 | 7.20 | 92% | 530K | 115 | 10051 | 70.13 |
| 5 | 5.4 | 302 | 13560 | 22,227 | 6.10 | 88% | 449K | 90 | 7700 | 72.9 |
| 6 | 5.4 | 302 | 13560 | 22,227 | 7.08 | 92% | 522K | 130 | 10000 | 73.6 |
| 7 | 8.1 | 453 | 20340 | 22,222 | 9.20 | 95% | 452K | 200 | 16815 | 92.75 |

As seen in Table 1, multiple bioreactors were seeded with different number of cells ranging 151 to 453 million cells per bioreactor. Three identical bioreactors (bioreactor #1, #2, and #3) had the same packed bed height (2.7 cm), were seeded with the same number of cells (151 million cells per bioreactor), and had the same total packed bed surface area (6780 cm²) and seeding density (22,222 cells/cm²). Three other identical bioreactors (#4, #5, and #6) had the same packed bed height (5.4 cm), were seeded with the same number of cells (302 million cells per bioreactor), and had the same total packed bed surface area (13,560 cm²) and seeding density (22,227 cells/cm²). A final bioreactor (#7) had an increased bed height (8.1 cm), total cells seeded (453 million cells), and packed bed surface area (20,340 cm²), but a similar seeding density (22,222 cells/cm²). During the five-day culture process, bulk media conditions (pH, DO₂, temperature, and CO₂) were maintained in automatic mode by a control system according to one or more embodiments described herein. The control system operated the media conditioning vessel to maintain media conditions, with FIGS. 4A-4C representing typical measurements of the controlled media. The bioreactor system's media perfusion flow rate was maintained automatically to maintain DO₂ at the bioreactor outlet at a specific saturation level. Again, the graph shown in FIG. 3 is typical of the perfusion flow and media outlet DO₂ found during these experiments. From graphs such as those in FIG. 3, the value of total oxygen consumption was derived, similar to that shown in FIG. 5. The slope of the linear curve fit of those graphs (like a in FIG. 5) was determined for each bioreactor run and is presented in last column of Table 1 (slope α). The value of the slope α determined as described above can be used as process analytical tool to control and describe upstream bioprocess and predict the biomass production inside the packed bed matrix.

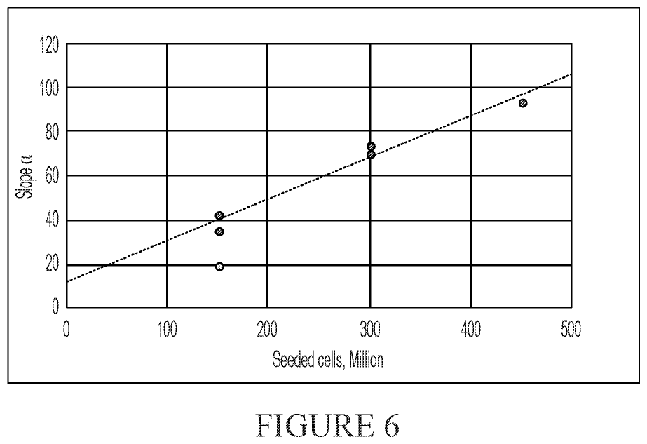

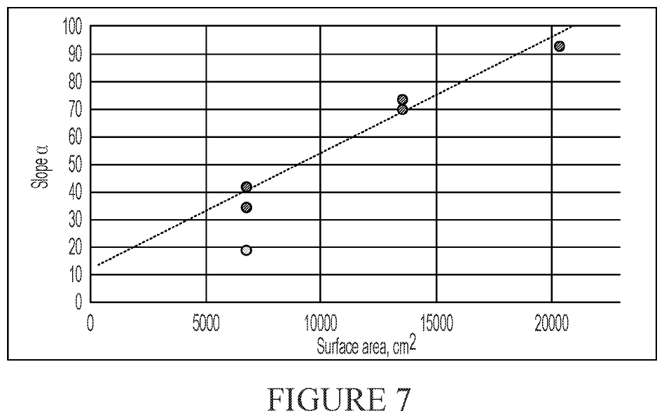

For example, FIGS. 6, 7, and 8 plot the values of α in Table 1 against the seeded cell number, packed bed surface area, and harvest density, respectively. The linearity of these graphs can be used to predict cell culture response according to various cell culture system parameters. For instance, the linearity of the graph in FIG. 6 indicates that upstream processes developed for small scale bioreactors #1 and #2 in Table 1 can be scaled 2× and 3× for bioreactors #4-7. Thus, constant monitoring and logging of the slope α value can serve for determining scalability of the upstream process. An alternative way to verify process scalability is to plot slope α relative to the surface area of bioreactor, as shown in FIG. 7. The orange data point in FIGS. 6 and 7 corresponds to failed bioreactor #3 from Table 1 (discussed below).

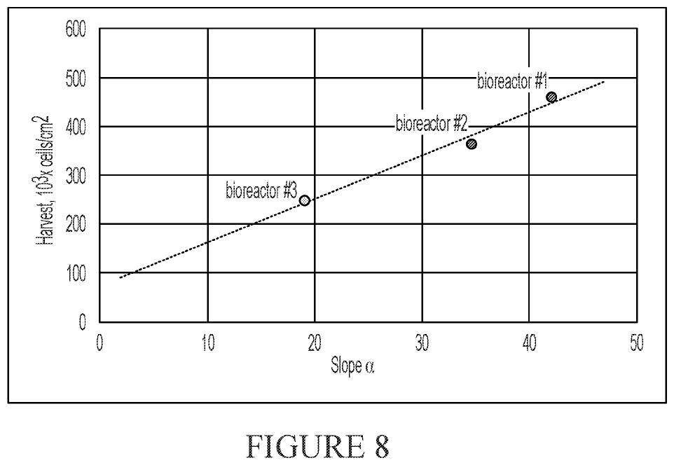

Monitoring of the slope α value during a bioreactor run serves as the characteristic signal signature that reflects health and expansion of cells culture. For example, as indicated in Table 1, bioreactors #1, 2 and 3 were seeded with the same number of cells. The characteristic signal signatures (slope α) were measured for all bioreactors. FIG. 8 indicates that real time monitoring of slope α can be used to compare performance of identical bioreactors and predict bioreactor productivity. From FIG. 8, it can be seen that bioreactor #3 run was in suboptimal conditions that resulted in lowest cells yield. Therefore, monitoring the value of slope α during bioprocess run can be used as characteristic signal signature for a given process and can detect any process deviation if the predetermined value is not within the range that was defined during process development optimization.

According to some embodiments, the media conditioning vessel is controlled by the controller to provide the proper temperature, pH, O₂, and nutrients. While in some embodiments, the bioreactor can also be controlled by the controller, in other embodiments the bioreactor is provided in a separate perfusion circuit, where a pump is used to control the flow rate of media through the perfusion circuit based on the detection of O2 at or near the outlet of the bioreactor.

The cell culture matrix can be arranged in multiple configurations within the culture chamber depending on the desired system. For example, in one or more embodiments, the system includes one or more layers of the substrate with a width extending across the width of a defined cell culture space in the culture chamber. Multiple layers of the substrate may be stacked in this way to a predetermined height. The substrate layers may be arranged such that the first and second sides of one or more layers are perpendicular to a bulk flow direction of culture media through the defined culture space within the culture chamber, or the first and second sides of one or more layers may be parallel to the bulk flow direction. In one or more embodiments, the cell culture matrix includes one or more substrate layers at a first orientation with respect to the bulk flow, and one or more other layers at a second orientation that is different from the first orientation. For example, various layers may have first and second sides that are parallel or perpendicular to the bulk flow direction, or at some angle in between.

In one or more embodiments, the cell culture system includes a plurality of discrete pieces of the cell culture substrate in a packed bed configuration, where the length and or width of the pieces of substrate are small relative to the culture chamber. As used herein, the pieces of substrate are considered to have a length and/or width that is small relative to the culture chamber when the length and/or width of the piece of substrate is about 50% or less of the length and/or width of the culture space. Thus, the cell culture system may include a plurality of pieces of substrate packed into the culture space in a desired arrangement. The arrangement of substrate pieces may be random or semi-random, or may have a predetermined order or alignment, such as the pieces being oriented in a substantially similar orientation (e.g., horizontal, vertical, or at an angle between 0° and 90° relative to the bulk flow direction).

The “defined culture space,” as used herein, refers to a space within the culture chamber occupied by the cell culture matrix and in which cell seeding and/or culturing is to occur. The defined culture space can fill approximately the entirety of the culture chamber, or may occupy a portion of the space within the culture chamber. As used herein, the “bulk flow direction” is defined as a direction of bulk mass flow of fluid or culture media through or over the cell culture matrix during the culturing of cells, and/or during the inflow or outflow of culture media to the culture chamber.

In some embodiments of this disclosure, there is only a single bulk flow direction within the defined culture space, the packed bed, and/or the bioreactor vessel, such that the liquid or media flow proceeds in predominately one direction from the bioreactor inlet through the packed bed to the bioreactor outlet. The liquid or media flow is uninterrupted by any complicated flow paths within the packed bed space and proceeds through the packed bed in predominately one direction. This avoids complicating flow paths used in some conventional bioreactors where flow spacers, separators, or channels are used to help distribute cell culture media through a cell culture substrate, often because of the inherent non-uniformity of the bioreactor or cell culture substrate. However, in embodiments of the current disclosure, such complicated flow paths are not necessary, and the media flow can be maintained in a single direction from the inlet of the bioreactor to the outlet of the bioreactor. The foregoing is not intended to preclude the use of flow distributor plates at the inlet and outlets of the bioreactor plate, which can be used to distribute fluid across a width of the bioreactor vessel and/or control pressure differentials within the reactor, but do not otherwise affect the bulk flow direction through the packed bed and/or within the cell culture space within the bioreactor vessel interior.

The packed bed cell culture matrix of one or more embodiments can include a substrate material constructed to have a uniform and ordered porous structure. The substrate may be referred to as a “structurally defined” substrate meaning that the substrate has a physical structure that is non-random, but instead is ordered according to defined parameters. In one or more embodiments, the structurally defined substrate includes a plurality of openings defining a porosity of the substrate, the plurality of openings being arrayed in a regular or uniform pattern in each substrate piece or layer. In one or more embodiments, the packed bed cell culture substrate may include a woven cell culture mesh substrate without any other form of cell culture substrate disposed in or interspersed with the cell culture matrix. That is, the woven cell culture mesh substrate of embodiments of this disclosure are effective cell culture substrates without requiring the type of irregular, non-woven substrates used in existing solution. This enables cell culture systems of simplified design and construction, while providing a high-density cell culture substrate with the other advantages discussed herein related to flow uniformity, harvestability, etc.

In one or more embodiments, a matrix is provided with a structurally defined surface area for adherent cells to attach and proliferate that has good mechanical strength and forms a highly uniform multiplicity of interconnected fluidic networks when assembled in a packed bed or other bioreactor. In particular embodiments, a mechanically stable, non-degradable woven mesh can be used as the substrate to support adherent cell production. The cell culture matrix disclosed herein supports attachment and proliferation of anchorage dependent cells in a high volumetric density format. Uniform cell seeding of such a matrix is achievable, as well as efficient harvesting of cells or other products of the bioreactor. In addition, the embodiments of this disclosure support cell culturing to provide uniform cell distribution during the inoculation step and achieve a confluent monolayer or multilayer of adherent cells on the disclosed matrix, and can avoid formation of large and/or uncontrollable 3D cellular aggregates with limited nutrient diffusion and increased metabolite concentrations. Thus, the matrix eliminates diffusional limitations during operation of the bioreactor. In addition, the matrix enables easy and efficient cell harvest from the bioreactor. The structurally defined matrix of one or more embodiments enables complete cell recovery and consistent cell harvesting from the packed bed of the bioreactor.

By using a structurally defined culture matrix of sufficient rigidity, high-flow-resistance uniformity across the matrix or packed bed is achieved. According to various embodiments, the matrix can be deployed in monolayer or multilayer formats. This flexibility eliminates diffusional limitations and provides uniform delivery of nutrients and oxygen to cells attached to the matrix. In addition, the open matrix lacks any cell entrapment regions in the packed bed configuration, allowing for complete cell harvest with high viability at the end of culturing. The matrix also delivers packaging uniformity for the packed bed, and enables direct scalability from process development units to large-scale industrial bioprocessing unit. The ability to directly harvest cells from the packed bed eliminates the need of resuspending a matrix in a stirred or mechanically shaken vessel, which would add complexity and can inflict harmful shear stresses on the cells. Further, the high packing density of the cell culture matrix yields high bioprocess productivity in volumes manageable at the industrial scale.

In contrast to existing cell culture substrates used in cell culture bioreactors (i.e., non-woven substrates of randomly ordered fibers), embodiments of this disclosure include a cell culture substrate having a defined and ordered structure. The defined and order structure allows for consistent and predictable cell culture results. In addition, the substrate has an open porous structure that prevents cell entrapment and enables uniform flow through the packed bed. This construction enables improved cell seeding, nutrient delivery, cell growth, and cell harvesting According to one or more particular embodiments, the matrix is formed with a substrate material having a thin, sheet-like construction having first and second sides separated by a relatively small thickness, such that the thickness of the sheet is small relative to the width and/or length of the first and second sides of the substrate. In addition, a plurality of holes or openings are formed through the thickness of the substrate. The substrate material between the openings is of a size and geometry that allows cells to adhere to the surface of the substrate material as if it were approximately a two-dimensional (2D) surface, while also allowing adequate fluid flow around the substrate material and through the openings. In some embodiments, the substrate is a polymer-based material, and can be formed as a molded polymer sheet; a polymer sheet with openings punched through the thickness; a number of filaments that are fused into a mesh-like layer; a 3D-printed substrate; or a plurality of filaments that are woven into a mesh layer. The physical structure of the matrix has a high surface-to-volume ratio for culturing anchorage dependent cells. According to various embodiments, the matrix can be arranged or packed in a bioreactor in certain ways discussed here for uniform cell seeding and growth, uniform media perfusion, and efficient cell harvest.

According to one or more embodiments the cell culture substrate can be one according to the cell culture matrices and/or substrate materials disclosed in U.S. patent application Ser. Nos. 16/781,685; 16/781,723; 16/781,764; 16/781,807; 16/781,847; 16/781,883; and 16/765,722, all of which are incorporated herein by reference in their entireties.

According to some embodiments, a method of cell culturing is also provided using bioreactors with the matrix for bioprocessing production of therapeutic proteins, antibodies, viral vaccines, or viral vectors.

The cell culture substrates and bioreactor systems provided offer numerous advantages. For example, the embodiments of this disclosure can support the production of any of a number of viral vectors, such as AAV (all serotypes) and lentivirus, and can be applied toward in vivo and ex vivo gene therapy applications. The uniform cell seeding and distribution maximizes viral vector yield per vessel, and the designs enable harvesting of viable cells, which can be useful for seed trains consisting of multiple expansion periods using the same platform. In addition, the embodiments herein are scalable from process development scale to production scale, which ultimately saves development time and cost. The methods and systems disclosed herein also allow for automation and control of the cell culture process to maximize vector yield and improve reproducibility. Finally, the number of vessels needed to reach production-level scales of viral vectors (e.g., 10¹⁶ to 10¹⁸ AAV VG per batch) can be greatly reduced compared to other cell culture solutions.

Embodiments are not limited to the vessel rotation about a central longitudinal axis. For example, the vessel may rotate about an axis that is not centrally located with respect to the vessel. In addition, the axis of rotation may be a horizonal or vertical axis.

### Definitions

“Wholly synthetic” or “fully synthetic” refers to a cell culture article, such as a microcarrier or surface of a culture vessel, that is composed entirely of synthetic source materials and is devoid of any animal derived or animal sourced materials. The disclosed wholly synthetic cell culture article eliminates the risk of xenogeneic contamination.

“Include,” “includes,” or like terms means encompassing but not limited to, that is, inclusive and not exclusive.

“Users” refers to those who use the systems, methods, articles, or kits disclosed herein, and include those who are culturing cells for harvesting of cells or cell products, or those who are using cells or cell products cultured and/or harvested according to embodiments herein.

“About” modifying, for example, the quantity of an ingredient in a composition, concentrations, volumes, process temperature, process time, yields, flow rates, pressures, viscosities, and like values, and ranges thereof, or a dimension of a component, and like values, and ranges thereof, employed in describing the embodiments of the disclosure, refers to variation in the numerical quantity that can occur, for example: through typical measuring and handling procedures used for preparing materials, compositions, composites, concentrates, component parts, articles of manufacture, or use formulations; through inadvertent error in these procedures; through differences in the manufacture, source, or purity of starting materials or ingredients used to carry out the methods; and like considerations. The term “about” also encompasses amounts that differ due to aging of a composition or formulation with a particular initial concentration or mixture, and amounts that differ due to mixing or processing a composition or formulation with a particular initial concentration or mixture.

“Optional” or “optionally” means that the subsequently described event or circumstance can or cannot occur, and that the description includes instances where the event or circumstance occurs and instances where it does not.

The indefinite article “a” or “an” and its corresponding definite article “the” as used herein means at least one, or one or more, unless specified otherwise.

Abbreviations, which are well known to one of ordinary skill in the art, may be used (e.g., “h” or “hrs” for hour or hours, “g” or “gm” for gram(s), “mL” for milliliters, and “rt” for room temperature, “nm” for nanometers, and like abbreviations).

Specific and preferred values disclosed for components, ingredients, additives, dimensions, conditions, and like aspects, and ranges thereof, are for illustration only; they do not exclude other defined values or other values within defined ranges. The systems, kits, and methods of the disclosure can include any value or any combination of the values, specific values, more specific values, and preferred values described herein, including explicit or implicit intermediate values and ranges.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that any particular order be inferred.

It will be apparent to those skilled in the art that various modifications and variations can be made without departing from the spirit or scope of the disclosed embodiments. Since modifications, combinations, sub-combinations and variations of the disclosed embodiments incorporating the spirit and substance of the embodiments may occur to persons skilled in the art, the disclosed embodiments should be construed to include everything within the scope of the appended claims and their equivalents.

1. A bioreactor system for culturing cells, the system comprising:
a cell culture vessel comprising at least one interior reservoir, an inlet fluidly connected to the reservoir, and an outlet fluidly connected to the reservoir:
a fluid flow path configured to supply fluid to the inlet and receive fluid from the outlet:
a media conditioning vessel fluidly connected to the cell culture vessel:
an outlet sensor arranged at or downstream of the outlet of the cell culture vessel;
wherein the outlet sensor is configured to detect a property of cell culture media exiting the cell culture vessel via the outlet, and
wherein the bioreactor system is configured to adjust a property of the cell culture media based on the property detected by the outlet sensor.

2. The bioreactor system of claim 1, wherein the property detected by the outlet sensor comprises at least one of pH, temperature, dissolved gas level, and nutrient level of the cell culture media, and
wherein the property of the cell culture media that is adjusted comprises at least one of pH, temperature, dissolved gas level, nutrient level, and flow rate of the cell culture media.

3. (canceled)

4. (canceled)

5. The bioreactor system of claim 1, further comprising a pump configured to control a flow rate in the fluid flow path.

6. The bioreactor system of claim 5, wherein the bioreactor system is configured to control the pump to adjust the flow rate based on the property detected by the outlet sensor.

7. The bioreactor system of claim 6, wherein the bioreactor system is configured to increase the flow rate when the amount of the dissolved gas decreases below a predetermined level.

8. The bioreactor system of claim 1, further comprising at least one media conditioning sensor configured to detect a media property in the media conditioning vessel, and
wherein the media property comprises at least one of dissolved gas level, pH, temperature, and nutrient level.

9-14. (canceled)

15. The bioreactor system of claim 1, further comprising a media conditioning control unit configured to control one or more media properties of the cell culture media.

16. The bioreactor system of claim 15, wherein the one or more media properties comprises dissolved gas level, pH, temperature, nutrient level, and flow rate.

17. The bioreactor system of claim 15, wherein the media conditioning control unit is arranged to receive a signal from the at least one media conditioning sensor and to control the at least one conditioning component based on the signal received.

18. The bioreactor system of claim 15, wherein the media conditioning control unit is configured to receive a signal from the outlet sensor and to control at least one of the at least one conditioning component and the pump based on the signal received from the outlet sensor.

19. The bioreactor system of claim 1, further comprising a cell culture substrate disposed in the reservoir and being configured for adhering cells thereto for cell culture, and
wherein the cell culture substrate is configured for uniform fluid flow therethrough.

20-29. (canceled)

30. The bioreactor system of claim 1,
wherein the cell culture vessel is configured for cell culture media to flow continuously from the inlet toward the outlet.

31. (canceled)

32. (canceled)

33. The bioreactor system of claim 1, wherein the outlet sensor is an in-line sensor.

34-44. (canceled)

45. A method of controlling a bioreactor system for culturing cells, comprising:
perfusing cell culture media through a cell culture vessel having an inlet through which the cell culture media enters the cell culture vessel and an outlet through which the cell culture media exists the cell culture vessel;
measuring an outlet property of the cell culture media using an outlet sensor at the outlet of the cell culture vessel; and
adjusting an inlet property of the cell culture media based on the property measured, the inlet property being a property of the cell culture media when entering the inlet

46. The method of claim 45, wherein the outlet property measured is at least one of pH, temperature, dissolved gas level, and nutrient level of the fluid, and
wherein the inlet property is at least one of pH, temperature, dissolved gas level, nutrient level, and flow rate of the cell culture media.

47. (canceled)

48. The method of claim 45, further comprising:
conditioning the cell culture media in a media conditioning vessel to adjust at least one of pH, temperature, dissolved gas level, and nutrient level of the cell culture media before it enters the cell culture vessel.

49. The method of claim 45, further comprising:
controlling a pump to adjusting the flow rate of cell culture media through the cell culture vessel.

50. The method of claim 45, further comprising:
measuring over time a level of dissolved oxygen in the cell culture media using the outlet sensor and calculating a rate of change of oxygen consumption by the cell culture over time; and
using the rate of change of oxygen consumption to determine a quality of health of the cell culture.

51. The method of claim 50, wherein the quality of health of the cell culture is a biomass production of the cell culture.

52. The method of claim 45, wherein the outlet sensor is at least one of integrated with or coupled to a tubing assembly attached to the outlet. or an in-line sensor.

53. (canceled)
